(19) United States
(12) Patent Application Publication
(10) Pub. No.: US 2023/0244873 A1
(43) Pub. Date: Aug. 3, 2023

(54) INFORMATION PROCESSING APPARATUS, INFORMATION PROCESSING METHOD, AND PROGRAM

(71) Applicant: FUJIFILM Corporation, Tokyo (JP)

(72) Inventor: Keigo NAKAMURA, Tokyo (JP)

(73) Assignee: FUJIFILM Corporation, Tokyo (JP)

(21) Appl. No.: 18/152,745

(22) Filed: Jan. 10, 2023

(30) Foreign Application Priority Data
Feb. 3, 2022 (JP) 2022-015957

(51) Int. Cl. G06F 40/289; G16H 50/20; G16H 30/40

(52) U.S. Cl. CPC G06F 40/289; G16H 50/20; G16H 30/40

(57) ABSTRACT

There are provided an information processing apparatus, an information processing method, and a program which appropriately perform processing relating to a key image to be associated with a series of sentences.

The information processing apparatus includes a processor; and a memory, and the information processing apparatus accepts a series of sentences including a diagnosis result of an image, specifies a relationship of two or more words included in the series of sentences, and decides at least one of necessity of association of a key image based on the image with the series of sentences or a candidate for the key image to be associated with the series of sentences on the basis of the relationship of the two or more words.

### CROSS-REFERENCE TO RELATED APPLICATION

The present application claims priority under 35 U.S.C § 119(a) to Japanese Patent Application No. 2022-015957 filed on Feb. 3, 2022, which is hereby expressly incorporated by reference, in its entirety, into the present application.

### BACKGROUND OF THE INVENTION

### 1. Field of the Invention

The present invention relates to an information processing apparatus, an information processing method, and a program, and particularly relates to a technique of analyzing a sentence including a diagnosis result of an image.

### 2. Description of the Related Art

A radiologist in the radiology department diagnoses medical images in response to an image diagnosis request from the attending physician of the medical department, and creates an interpretation report describing the presence or absence of abnormalities. In this case, attaching a key image to the interpretation report is a burden.

In order to address such a problem, JP2015-162082A discloses a system that extracts a registered word or synonym registered in a dictionary from a plurality of words constituting a character string in a case where the character string is input to a finding display region, and specifies an image relating to the extracted character string from a plurality of images relating to a patient as an interpretation target.

Further, JP2018-028562A discloses that in a system that generates an interpretation report by a voice input, in a case where a word set in advance is detected, a finding statement is generated on the basis of the recognized word history, and a slice image that was displayed in a case where the voice was spoken is associated with the finding statement.

### SUMMARY OF THE INVENTION

However, the techniques disclosed in JP2015-162082A and JP2018-028562A do not analyze the entire word string and voice input, and have a problem that an appropriate image may not be appropriately selected.

In the technique disclosed in JP2015-162082A, there is a problem that the image is always selected even in a case where the image is unnecessary.

The present invention is made in view of such circumstances, and an object thereof is to provide an information processing apparatus, an information processing method, and a program which appropriately performs processing relating to a key image associated with a series of sentences.

An aspect of an information processing apparatus for achieving the object is an information processing apparatus including at least one processor; and at least one memory that stores a command for the at least one processor to execute, in which the at least one processor is configured to accept a series of sentences including a diagnosis result of an image; specify a relationship of two or more words included in the series of sentences; and decide at least one of necessity of association of a key image based on the image with the series of sentences or a candidate for the key image to be associated with the series of sentences on the basis of the relationship of the two or more words. According to the aspect, at least one of the necessity of association of the key image with the series of sentences or the candidate for the key image to be associated with the series of sentences is decided, and therefore, the processing relating to the key image to be associated with the series of sentences can be appropriately performed.

Another aspect of the information processing apparatus for achieving the object may be an information processing apparatus including at least one processor; and at least one memory that stores a command for the at least one processor to execute, in which the at least one processor is configured to accept a series of sentences including a diagnosis result of an image; specify a relationship of two or more words included in the series of sentences; determine necessity of association of a key image based on the image with the series of sentences on the basis of the relationship of the two or more words; and decide a candidate for the key image to be associated with the series of sentences in a case where the necessity of the association is determined. Even in this aspect, the processing relating to the key image to be associated with the series of sentences can be appropriately performed.

The series of sentences includes one or a plurality of sentences.

It is preferable that the two or more words include at least two words from among a word representing a region of interest, a word representing facticity, a word representing change information, a word representing a position, a word representing a size, a word representing a characteristic, or a word representing an imaging condition.

It is preferable that the two or more words include a word representing a region of interest, and a word representing facticity of the region of interest, and the at least one processor is configured to determine that the association of the key image is necessary in a case where the facticity affirms existence of the region of interest; and determine that the association of the key image is not necessary in a case where the facticity denies the existence of the region of interest.

It is preferable that the word representing the change information includes a word representing change information on at least one of a size or an amount.

It is preferable that the at least one processor extracts the candidate for the key image from the image on the basis of the position.

It is preferable that the at least one processor is configured to accept two or more types of images of which the imaging conditions are different; and extract the candidate for the key image from the two or more types of images.

It is preferable that the image is a medical image, the two or more words include a word representing a disease name, and the at least one processor extracts the candidate for the key image on the basis of the disease name.

It is preferable that the image is a medical image, the two or more words include a word representing a region of interest, and a word representing a malignancy grade of the region of interest, and the at least one processor is configured to determine that the association of the key image is necessary in a case where the malignancy grade affirms malignancy of the region of interest; and determine that the association of the key image is not necessary in a case where the malignancy grade denies the malignancy of the region of interest.

It is preferable that the at least one processor is configured to display the candidate for the key image on a display, accept an operation by a user; and associate the candidate of the key image with the series of sentences, as the key image according to the operation.

It is preferable that the image is a three-dimensional image, and the at least one processor is configured to display, as the candidate for the key image, a slice image at any slice position of the three-dimensional image on a display; accept a change of the slice position of the candidate for the key image by a user; and associate the slice image at the changed slice position with the series of sentences, as the key image.

An aspect of an information processing method for achieving the object is an information processing method including an acceptance step of accepting a series of sentences including a diagnosis result of an image; a specifying step of specifying a relationship of the two or more words; and a decision step of deciding at least one of necessity of association of a key image based on the image with the series of sentences or a candidate for the key image to be associated with the series of sentences on the basis of the two or more words and the relationship. According to the aspect, at least one of the necessity of association of the key image with the series of sentences or the candidate for the key image to be associated with the series of sentences is decided, and therefore, the processing relating to the key image to be associated with the series of sentences can be appropriately performed.

An aspect of a program for achieving the object is a program for causing a computer to execute the information processing method described above. A computer-readable non-transitory storage medium in which the program is stored may be included in the aspect. According to the aspect, at least one of the necessity of association of the key image with the series of sentences or the candidate for the key image to be associated with the series of sentences is decided, and therefore, the processing relating to the key image to be associated with the series of sentences can be appropriately performed.

According to the present invention, the processing relating to the key image to be associated with the series of sentences can be appropriately performed.

### BRIEF DESCRIPTION OF THE DRAWINGS

FIG. 1 is an entire configuration diagram of a medical information processing system.

FIG. 2 is a block diagram illustrating a configuration of a medical information processing apparatus.

FIG. 3 is a flowchart illustrating a medical information processing method using the medical information processing system.

FIG. 4 is a diagram illustrating an interpretation image and a description example of an interpretation report.

FIG. 5 is a diagram illustrating a structured result obtained by structuring a finding statement of the interpretation report by natural language processing.

FIG. 6 is a diagram illustrating two key images extracted from an image.

FIG. 7 is a diagram illustrating an interpretation image and a description example of an interpretation report.

FIG. 8 is a diagram illustrating a structured result of a finding statement of an interpretation report.

FIG. 9 is a diagram illustrating an interpretation result.

FIG. 10 is a diagram illustrating an interpretation result.

FIG. 11 is a diagram illustrating an interpretation result.

FIG. 12 is a diagram illustrating an interpretation result.

FIG. 13 is a diagram illustrating an interpretation result.

FIG. 14 is a diagram illustrating an interpretation result.

FIG. 15 is a diagram for describing the structuring of a finding statement.

FIG. 16 is a diagram for describing the structuring of a finding statement.

FIG. 17 is a diagram for describing the structuring of a finding statement.

### DESCRIPTION OF THE PREFERRED EMBODIMENTS

Hereinafter, preferred embodiments of the present invention will be described in detail with reference to the accompanying drawings.

Medical Image Processing System

A medical information processing system according to the present embodiment is a system that captures a medical image of a subject (patient), accepts a finding statement (an example of a “series of sentences”) including a diagnosis result of the captured medical image, analyzes the entire accepted finding statement, and performs processing relating to a key image associated with the finding statement on the basis of the analyzed result.

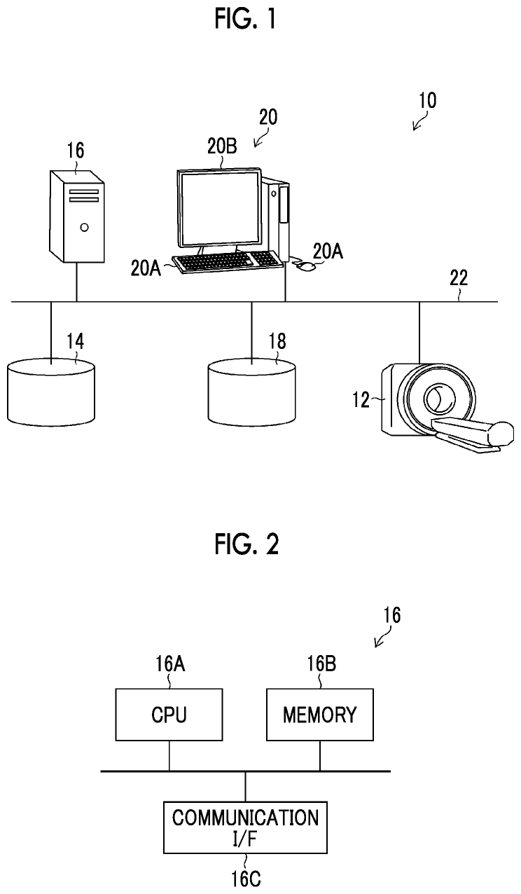

FIG. 1 is an entire configuration diagram of a medical information processing system 10. As illustrated in FIG. 1, the medical information processing system 10 includes a medical image examination device 12, a medical image database 14, a medical information processing apparatus 16, an interpretation report database 18, and a user terminal 20.

The medical image examination device 12, the medical image database 14, the medical information processing apparatus 16, the interpretation report database 18, and the user terminal 20 are connected via a network 22 to transmit and receive data to and from each other. The network 22 includes a wired or wireless local area network (LAN) for communication connection of various devices in the medical institution. The network 22 may include a wide area network (WAN) that connects LANs of a plurality of medical institutions.

The medical image examination device 12 is an imaging device that images an examination target part of a subject and generates a medical image. Examples of the medical image examination device 12 include an X-ray imaging device, a computed tomography (CT) device, a magnetic resonance imaging (MRI) device, a positron emission tomography (PET) device, an ultrasound device, and a computed radiography (CR) device using flat X-ray detector.

The medical image database 14 is a database that manages the medical image captured by the medical image examination device 12. As the medical image database 14, a computer including a large-capacity storage device for storing the medical image is applied. Software providing a function of a database management system is incorporated in the computer.

As a format of the medical image, Digital Imaging and Communications in Medicine (Dicom) standards can be applied. The medical image may be added with accessory information (Dicom tag information) defined in the Dicom standards. The term “image” used in this specification includes not only the image itself, such as a photograph, but also image data which is a signal representing an image.

The medical information processing apparatus 16 is a device that decides at least one of the necessity of associating the key image with the finding statement or candidates for the key image to be associated with the finding statement. As the medical information processing apparatus 16, a personal computer or a workstation (an example of a “computer”) can be applied. FIG. 2 is a block diagram illustrating a configuration of the medical information processing apparatus 16. As illustrated in FIG. 2, the medical information processing apparatus 16 includes a processor 16A, a memory 16B, and a communication interface 16C.

The processor 16A executes a command stored in the memory 16B. The hardware structures of the processor 16A are the following various processors. The various processors include a central processing unit (CPU) as a general-purpose processor executing software (program) and acting as various functional units, a graphics processing unit (GPU) as a processor specialized for image processing, a programmable logic device (PLD) as a processor of which the circuit configuration can be changed after manufacturing such as a field programmable gate array (FPGA), a dedicated electrical circuit or the like as a processor having a circuit configuration designed exclusively for executing specific processing such as an application specific integrated circuit (ASIC).

One processing unit may be configured by one of the various processors, or configured by the same or different kinds of two or more processors (for example, combination of a plurality of FPGAs, combination of the CPU and the FPGA, combination of the CPU and the GPU, or the like). In addition, a plurality of functional units may be configured by one processor. As an example where a plurality of functional units are formed by one processor, first, there is an aspect where one processor is formed of a combination of one or more CPUs and software as typified by a computer, such as a client or a server, and this processor acts as a plurality of functional units. Second, there is a form where a processor fulfilling the functions of the entire system including a plurality of functional units by one integrated circuit (IC) chip as typified by a system on chip (SoC) or the like is used. In this manner, various functional units are configured by using one or more of the above-described various processors as hardware structures.

Furthermore, the hardware structures of these various processors are more specifically electrical circuitry where circuit elements, such as semiconductor elements, are combined.

The memory 16B stores a command for the processor 16A to execute. The memory 16B includes a random access memory (RAM) and a read only memory (ROM) (not illustrated). The processor 16A uses the RAM as a work area, executes software using various parameters and programs including a medical information processing program described later, which are stored in the ROM, and executes various kinds of processing of the medical information processing apparatus 16 by using the parameters stored in the ROM or the like.

The communication interface 16C controls communication with the medical image examination device 12, the medical image database 14, the interpretation report database 18, and the user terminal 20 via the network 22 according to a predetermined protocol.

The medical information processing apparatus 16 may be a cloud server that can be accessed from a plurality of medical institutions via the Internet. The processing performed in the medical information processing apparatus 16 may be a billing or fixed fee cloud service.

Returning to the description of FIG. 1, the interpretation report database 18 is a database that manages an interpretation report generated by a user such as a radiologist in the user terminal 20. The interpretation report includes a finding statement. The finding statement is not limited to a sentence delimited by a punctuation mark, a period, or the like, and may be a group of words. The finding statement may be one sentence, or may be a series of sentences including a plurality of sentences. The interpretation report may include a key image associated with the finding statement.

As the interpretation report database 18, a computer including a large-capacity storage device for storing the interpretation report is applied. Software providing a function of a database management system is incorporated in the computer. The medical image database 14 and the interpretation report database 18 may be configured by one computer.

The user terminal 20 is a terminal device for the user to view and edit the interpretation report. As the user terminal 20, for example, a personal computer is applied. The user terminal 20 may be a workstation, or may be a tablet terminal. The user terminal 20 includes an input device 20A and a display 20B. The user inputs an instruction to the medical information processing system 10 by using the input device 20A. The user terminal 20 displays the medical image and the interpretation report on the display 20B. Further, the user interprets (an example of “diagnosis”) the medical image displayed on the display 20B, and inputs the finding statement as the interpretation result (an example of a “diagnosis result”) using the input device 20A.

Medical Information Processing Method

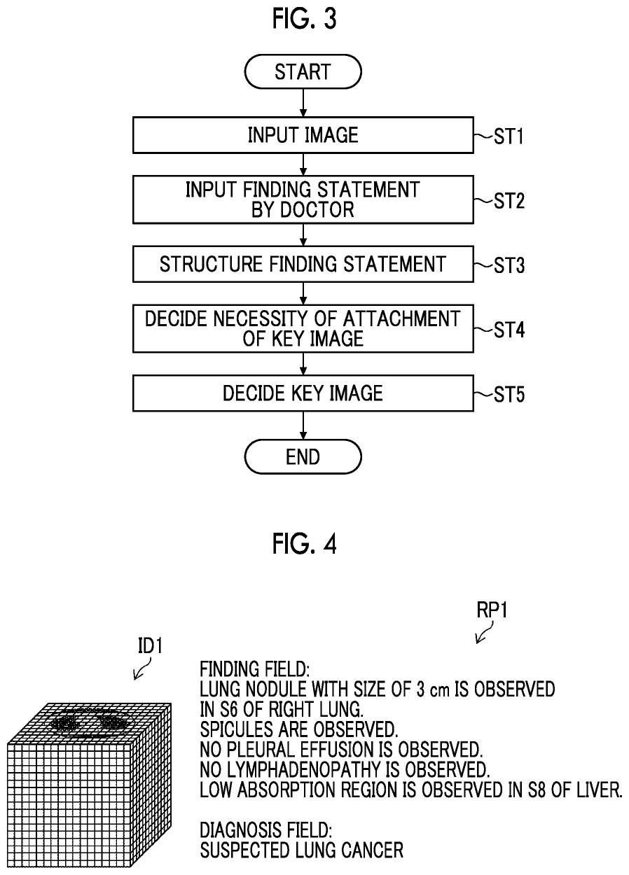

FIG. 3 is a flowchart illustrating a medical information processing method using the medical information processing system 10. The medical information processing method is realized by the processor 16A executing the medical information processing program stored in the memory 16B. The medical information processing program may be provided by a computer-readable non-transitory storage medium. In this case, the medical information processing apparatus 16 may read the medical information processing program from the non-transitory storage medium, and store the information processing program in the memory 16B.

In an image input step in Step ST1, the medical image, which is captured by the medical image examination device 12 and is stored in the medical image database 14, is transmitted to the user terminal 20 operated by the user. The medical information processing apparatus 16 receives (an example of “accept”) the medical image by the user terminal 20, and displays the received medical image on the display 20B. Thereby, the user can interpret the medical image displayed on the display 20B.

In a finding statement input step in Step ST2, the user generates the finding statement including the interpretation result for the medical image displayed on the display 20B in the image input step, and inputs the finding statement to the user terminal 20 using the input device 20A.

In a finding statement structuring step in Step ST3, the medical information processing apparatus 16 accepts (an example of “acceptance step”) the finding statement input in the finding statement input step. The acceptance of the finding statement may be performed simultaneously with the finding statement input step (in real time), or the past finding statement obtained in the finding statement input step performed in the past may be accepted.

Further, in the finding statement structuring step, the medical information processing apparatus 16 structures the accepted finding statement using the known natural language processing (an example of “extraction step”, an example of “specifying step”), and acquires the structured result. The natural language processing is a technology of allowing a computer to process natural languages used in daily life, and is processing including morphological analysis of decomposing a sentence into words, syntactic analysis of analyzing relationships between words obtained by the morphological analysis and building a syntax tree illustrating the structure of dependencies between words, and the like. By the structuring, the medical information processing apparatus 16 can extract two or more words from the finding statement, and specify the relationship of two or more words.

In an attachment necessity decision step (an example of a “decision step”) in Step ST4, the medical information processing apparatus 16 performs processing of determining the necessity of attachment (an example of “association”) of the key image to the finding statement on the basis of the structured result in the finding statement structuring step. The key image is an image which is determined to be important for the interpretation based on the content of the finding statement, among the medical images input in the image input step.

In a case where it is determined in the attachment necessity decision step that the association of the key image with the finding statement is not necessary, the processing of the present flowchart is ended. That is, the association of the key image with the finding statement is not performed. On the other hand, in a case where it is determined in the attachment necessity decision step that the association of the key image with the finding statement is necessary, the medical information processing apparatus 16 executes the processing of Step ST5.

In the key image decision step (an example of a “decision step”) in Step ST5, the medical information processing apparatus 16 analyzes the medical image input in the image input step and decides the key image to be associated with the finding statement on the basis of the structured result in the finding statement structuring step. Further, the medical information processing apparatus 16 associates the decided key image with the finding statement.

The finding statement and the key image associated with the finding statement may be stored in the interpretation report database 18 as one interpretation report. Only the finding statement may be stored in the interpretation report database 18 as the interpretation report, and the key image associated with the finding statement may be associated with the interpretation report by hyperlinking to the medical image database 14 or the like.

The medical information processing apparatus 16 may analyze the medical image on the basis of the structured result of the finding statement, decide the candidates for the key image to be associated with the finding statement, and allow the user to check the candidates for the key image. For example, the medical information processing apparatus 16 displays the candidates for the key image on the display 20B. The user checks the candidates for the key image displayed on the display 20B, and inputs an operation of confirming the candidate for the key image as the key image using the input device 20A in a case where there is no problem. By this operation, the medical information processing apparatus 16 associates the candidate of the key image as the key image with the finding statement.

Further, the medical information processing apparatus 16 may decide candidates for a plurality of key images, and display the candidates for the plurality of key images on the display 20B so that the user can select the candidate. In this case, the user selects at least one key image using the input device 20A, from the candidates for the plurality of key images displayed on the display 20B. The medical information processing apparatus 16 associates the selected candidate of the key image as the key image with the finding statement.

The medical information processing apparatus 16 may display a slice image at any slice position of a three-dimensional image as the candidate for the key image on the display 20B. In this case, the user may change the slice position of the candidate for the key image using the input device 20A. The medical information processing apparatus 16 may accept the change of the slice position by the user, and associate the slice image at the changed slice position as the key image with the finding statement.

In this manner, with the medical information processing method, it is possible to decide the necessity of the association of the key image with the finding statement. Further, with the medical information processing method, in a case where the association of the key image with the finding statement is necessary, it is possible to decide the candidate for the key image to be associated with the finding statement. With the medical information processing method, it is possible to associate the key image with the finding statement.

In the medical information processing method, the attachment necessity decision step may be omitted. That is, the medical information processing apparatus 16 may decide the key image to be associated with the finding statement without determining the necessity of the association of the key image with the finding statement.

Hereinafter, the medical information processing method will be described with more specific examples.

### First Example

An example in which the user interprets a three-dimensional chest and abdomen CT image will be described. FIG. 4 is a diagram illustrating an interpretation image and a description example of the interpretation report. A diagnostic image ID1 as the interpretation image is a three-dimensional CT image of an interpretation target. That is, the diagnostic image ID1 is volume data in which voxels are arranged three-dimensionally and a CT value is stored in each voxel.

An interpretation report RP1 includes a finding field and a diagnosis field. The finding field is a field in which the finding for the diagnostic image ID1 is described by the user. Further, the diagnosis field is a field in which the diagnosis for the diagnostic image ID1 is described by the user. Note that the finding statement in the present embodiment includes a sentence described in the finding field, and a sentence described in the diagnosis field.

As illustrated in FIG. 4, the finding field of the interpretation report RP1 describes “A lung nodule with a size of 3 cm is observed in S6 of the right lung. Spicules are observed. No pleural effusion is observed. No lymphadenopathy is observed. A low absorption region is observed in S8 of the liver”. Further, the diagnosis field of the interpretation report RP1 describes “suspected lung cancer”.

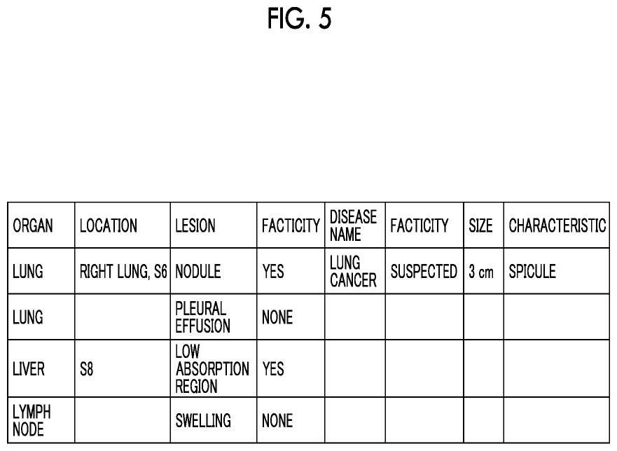

FIG. 5 is a diagram illustrating a structured result obtained by structuring the finding statement of the interpretation report RP1 by natural language processing. As illustrated in FIG. 5, in the structured result, the finding statement described in free description is structured as information for each lesion.

Here, the relationship is specified and classified such that “lung” is in the item of “organ”, “right lung” and “S6” are in the item of “location”, “nodule” is in the item of “lesion”, “yes” is in the item of “facticity of lesion”, “lung cancer” is in the item of “disease name”, “suspected” is in the item of “facticity of disease name”, “3 cm” is in the item of “size”, and “spicule” is in the item of “characteristic”. The facticity means the possibility of existence.

Similarly, the relationship is specified and classified such that “lung” is in the item of “organ”, “pleural effusion” is in the item of “lesion”, and “none” is in the item of “facticity of lesion”. The relationship is specified and classified such that “liver” is in the item of “organ”, “S8” is in the item of “location”, “low absorption region” is in the item of “lesion”, and “yes” is in the item of “facticity of lesion”. Further, the relationship is specified and classified such that “lymph node” is in the item of “organ”, “swelling” is in the item of “lesion”, and “none” is in the item of “facticity of lesion”.

In this manner, in the structured result, two or more words extracted from the finding statement and the relationship thereof are specified. Two or more words extracted from the finding statement include a word representing an organ, a word representing a location (position), a word representing a lesion, a word representing the facticity of the lesion, a word representing a disease name, a word representing the facticity of the disease name, a word representing a size, and a word representing a characteristic.

The medical information processing apparatus 16 decides the necessity of association of the key image with the finding statement on the basis of the two or more words and the relationship thereof. For example, in a case where the facticity affirms the existence of the region of interest, the medical information processing apparatus 16 determines that the association of the key image is necessary, and in a case where the facticity denies the existence of the region of interest, the medical information processing apparatus 16 determines that the association of the key image is not necessary. The region of interest is, for example, a lesion, an organ (overall deformation or the like), an anatomical region, or an image feature region (low absorption region or the like).

In the example of the structured result illustrated in FIG. 5, since the facticity affirms the existence of “nodule” and “low absorption region” of the lesion as the region of interest, the medical information processing apparatus 16 determines that the association of the key image is necessary.

In a case where it is determined that the association of the key image is necessary, the medical information processing apparatus 16 recognizes the organ and the lesion from the diagnostic image ID1 by known image processing on the basis of the two or more words and the relationship, and automatically decides the key image to be associated with the finding statement.

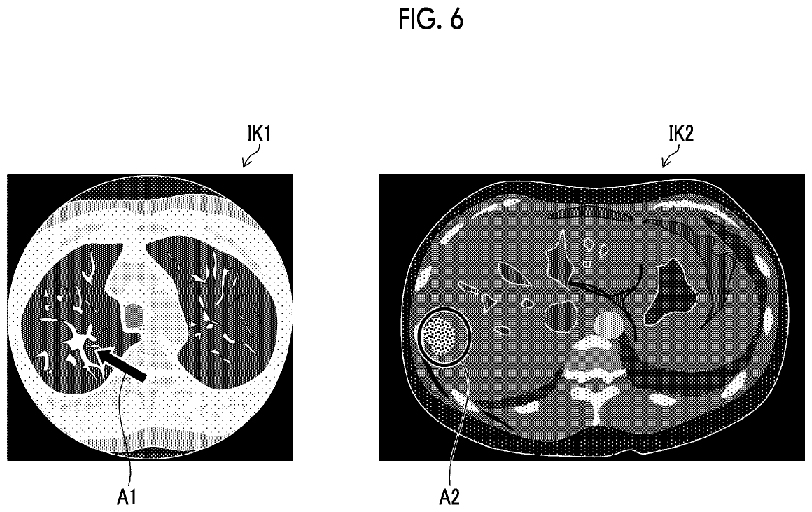

FIG. 6 is a diagram illustrating two key images IK1 and IK2 extracted from the diagnostic image ID1. By the structured result illustrated in FIG. 5, it can be seen that the lesions are present at two locations of “nodule” of “lung” and “low absorption region” of “liver”. Accordingly, the medical information processing apparatus 16 recognizes “nodule” of “lung” and “low absorption region” of “liver” from the diagnostic image ID1, and automatically decides the key images IK1 and IK2 to be associated with the finding statement.

In a case where the region of interest is a lesion, the lesion is extracted by known computer-aided diagnosis (CAD), and a slice image in which the lesion is most conspicuously shown may be used as the key image.

In a case where the region of interest is an abnormality in an organ, such as the uneven contour in cirrhosis of the liver, the liver is extracted by organ extraction/labeling processing, and a slice image at a slice position where the widest area is shown may be used as the key image.

A multiplanar reconstruction (MPR) image different from the slice plane or a volume rendering image may be created on the basis of a predetermined rule, and used as the key image, as necessary.

In the example illustrated in FIG. 6, an arrow annotation A1 is added to a region of the nodule in the key image IK1 of the lung, and a round frame annotation A2 is added to a region of the low absorption region in the key image IK2 of the liver. In this manner, the medical information processing apparatus 16 may perform processing of clearly indicating the region of the region of interest in the key image.

### Second Example

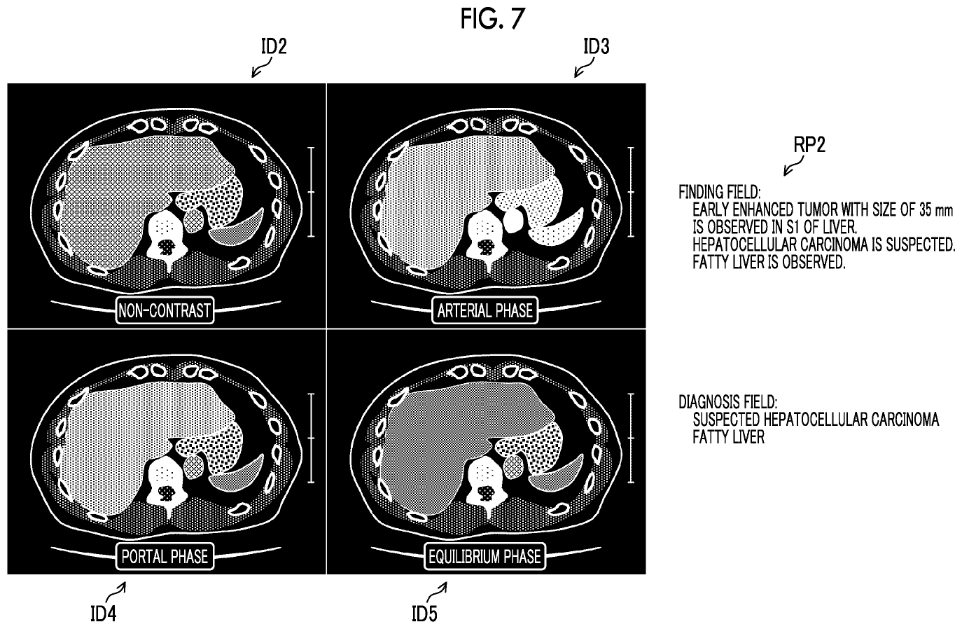

An example in which the user interprets an image of a dynamic CT examination of the liver will be described. FIG. 7 is a diagram illustrating the interpretation image and a description example of the interpretation report. Diagnostic images ID2, ID3, ID4, and ID5 are sectional images of the dynamic CT examination, each of which is to be interpreted. The imaging time phases of the diagnostic images ID2, ID3, ID4, and ID5 are “non-contrast”, “arterial phase”, “portal phase”, and “equilibrium phase”, respectively. In this manner, in the second example, the medical information processing apparatus 16 accepts two or more types of medical images having different imaging time phases, and displays the images on the display 20B.

An interpretation report RP2 includes a finding field and a diagnosis field as in the interpretation report RP1. The finding field describes “An early enhanced tumor with a size of 35 mm is observed in S1 of the liver. A hepatocellular carcinoma is suspected. A fatty liver is observed”. Further, the diagnosis field describes “suspected hepatocellular carcinoma” and “fatty liver”.

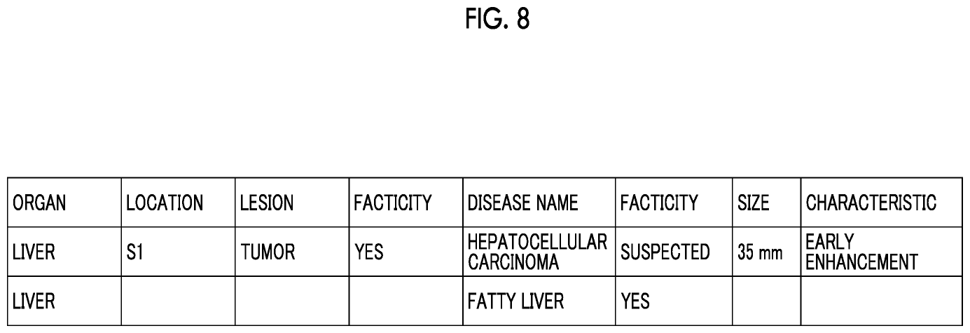

FIG. 8 is a diagram illustrating the structured result of the finding statement of the interpretation report RP2. The medical information processing apparatus 16 decides the key image on the basis of the structured result illustrated in FIG. 8. The hepatocellular carcinoma has a feature of being stained white in the arterial phase and being dark in the equilibrium phase. Accordingly, the medical information processing apparatus 16 decides the key image from the images of “arterial phase” in a case of “tumor”. Further, “fatty liver” appears whiter than usual in the images of “non-contrast”, and thus is not diagnosed in the contrast-enhanced images. Accordingly, the medical information processing apparatus 16 decides the key image from the images of “non-contrast” in a case of “fatty liver”. In this manner, the medical information processing apparatus 16 can determine the type of the image to be used as the key image from the disease name.

### Third Example

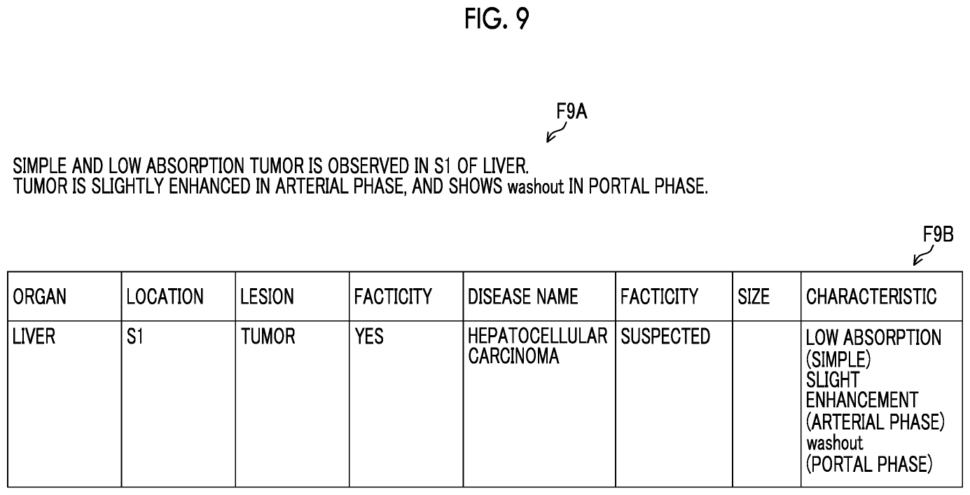

An example in which the user interprets an image of the dynamic CT examination of the liver different from the second example will be described. FIG. 9 is a diagram illustrating an interpretation result. F9A illustrated in FIG. 9 is a finding statement of the interpretation report. As illustrated in F9A, the finding statement describes “A simple and low absorption tumor is observed in S1 of the liver. The tumor is slightly enhanced in the arterial phase, and shows washout in the portal phase”.

F9B illustrated in FIG. 9 is the structured result of the finding statement of F9A. As illustrated in F9B, as a result of the structuring, the relationship is specified and classified such that “tumor” is in the item of “lesion”, “low absorption (simple)”, “slight enhancement (arterial phase)”, and “washout (portal phase)” are in the item of “characteristic”. That is, for the tumor, it is extracted that the low absorption is observed in the simple CT, and slight enhancement is observed in the arterial phase, and washout is observed in the portal phase.

In this manner, two or more words extracted from the finding statement include a word representing the imaging time phase (an example of “imaging condition”).

The medical information processing apparatus 16 decides the key image from the images of “simple” for “low absorption”, decides the key image from the images of “arterial phase” for “slight enhancement”, and decides the key image from the images of “portal phase” for “washout” on the basis of the structured result illustrated in F9B. Here, the disease name is unknown, the medical information processing apparatus 16 decides a plurality of images as the key image.

In this manner, the medical information processing apparatus 16 accepts two or more types of medical images having different imaging time phases, and extracts candidates for the key image from the two or more types of medical images.

### Fourth Example

An example in which the user interprets an image of a follow-up examination of the lung nodule will be described. The follow-up examination is an examination performed on the same subject after a certain period has elapsed in order for the doctor to check the progress of the subject.

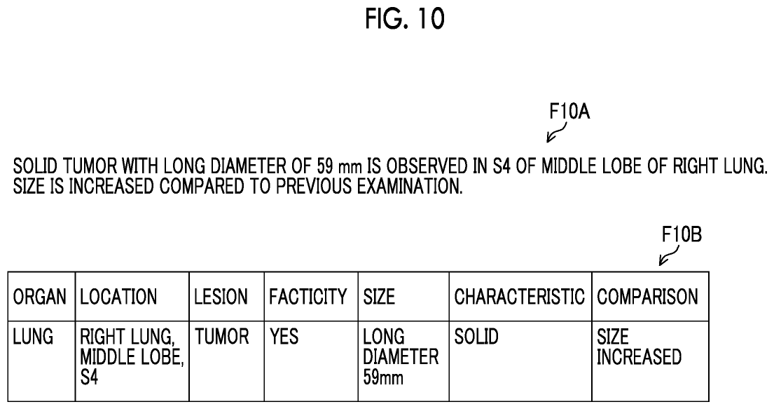

FIG. 10 is a diagram illustrating an interpretation result. F10A illustrated in FIG. 10 is a finding statement of the interpretation report. As illustrated in F10A, the finding statement describes “A solid tumor with a long diameter of 59 mm is observed in S4 of the middle lobe of the right lung. The size is increased compared to the previous examination”.

F10B illustrated in FIG. 10 is the structured result of the finding statement of F10A. As illustrated in F10B, as a result of the structuring, the relationship is specified and classified such that “tumor” is in the item of “lesion”, and “size increased” is in the item of “comparison”. The medical information processing apparatus 16 decides that the association of the key image with the finding statement is necessary on the basis of “size increased” of the structured result. The medical information processing apparatus 16 may decide a slice image at the same slice position as the key image (key image associated with the finding statement of the previous examination) of the interpretation report of the previous examination, as the key image.

Here, a case where the size is increased has been exemplified, but the medical information processing apparatus 16 decides that the association of the key image with the finding statement is necessary also in a case where the size is decreased. Further, in a case of the follow-up examination in which the lesion is “pleural effusion”, the medical information processing apparatus 16 decides that the association of the key image with the finding statement is necessary in a case where the storage amount is increased or decreased.

In this manner, the word to be extracted from the finding statement includes a word representing the comparison (an example of “change information”), and the word representing the comparison includes a word representing the comparison of at least one of the size or amount. Further, in a case where a word representing the comparison and a word representing the facticity are included in the finding statement and the relationship thereof is specified, it is decided that the association of the key image with the finding statement is necessary.

In a case where a word representing the region of interest in the past, a word representing the comparison, and a word representing the facticity are included in the finding statement and the relationship thereof is specified, it may be decided that the association of the key image with the finding statement is necessary.

In a case where the word to be extracted from the finding statement includes a word representing the comparison and a word representing the malignancy grade and the comparison indicates that the lesion is malignancy, it may be decided that the association of the key image with the finding statement is necessary.

### Fifth Example

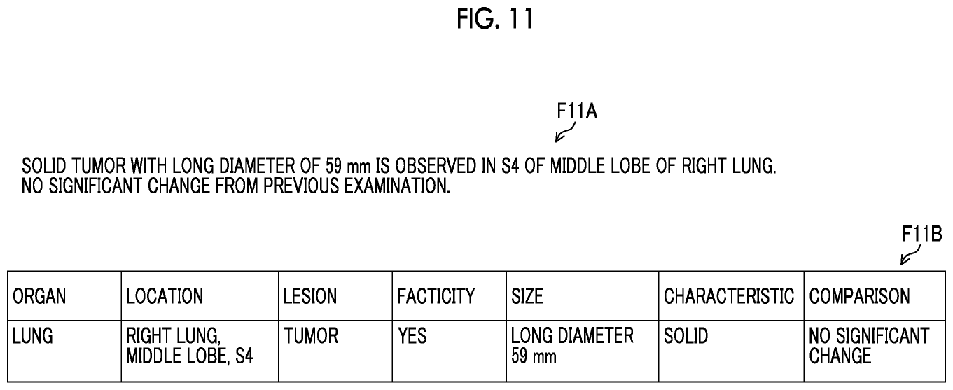

An example in which the user interprets an image of the follow-up examination of the lung nodule different from the fourth example will be described. FIG. 11 is a diagram illustrating an interpretation result. F11A illustrated in FIG. 11 is a finding statement of the interpretation report. As illustrated in F11A, the finding statement describes “A solid tumor with a long diameter of 59 mm is observed in S4 of the middle lobe of the right lung. No significant change from the previous examination”.

F11B illustrated in FIG. 11 is the structured result of the finding statement of F11A. As illustrated in F11B, as a result of the structuring, the relationship is specified and classified such that “tumor” is in the item of “lesion”, and “no significant change” is in the item of “comparison”. The medical information processing apparatus 16 decides that the association of the key image with the finding statement is not necessary on the basis of the structured result of “no significant change”.

In this manner, the medical information processing apparatus 16 decides that the association of the key image with the finding statement is not necessary in a case where the word representing the comparison denies change over time. Even in a case where the size of the lesion is not changed, it may be decided that the association of the key image with the finding statement is necessary.

### Sixth Example

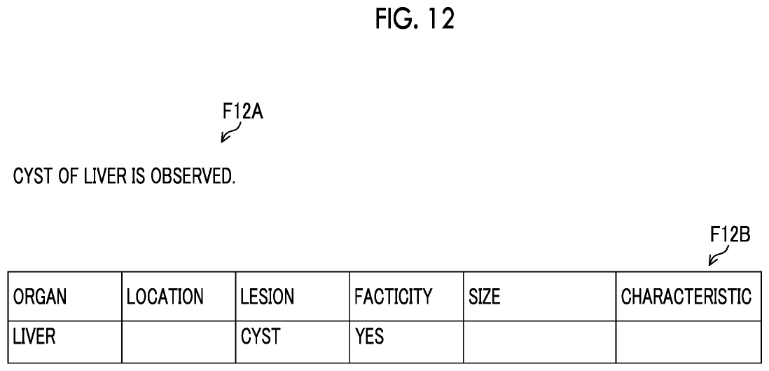

An example in which a liver cyst is found as a result of the interpretation will be described. FIG. 12 is a diagram illustrating an interpretation result. F12A illustrated in FIG. 12 is a finding statement of the interpretation report. As illustrated in F12A, the finding statement describes “A cyst of the liver is observed”.

F12B illustrated in FIG. 12 is the structured result of the finding statement of F12A. As illustrated in F12B, as a result of the structuring, the relationship is specified and classified such that “liver” is in the item of “organ”, “cyst” is in the item of “lesion”, and “yes” is in the item of “facticity”. Since the liver cyst clinically does not require treatment intervention, the medical information processing apparatus 16 decides that the association of the key image with the finding statement is not necessary on the basis of the structured result.

In this manner, the medical information processing apparatus 16 decides that the association of the key image with the finding statement is not necessary in a case where the word representing the lesion denies the treatment intervention.

### Seventh Example

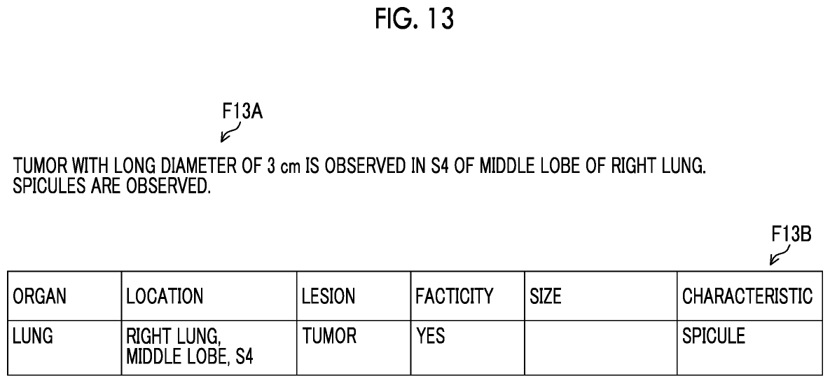

An example in which a lung nodule of which the characteristic indicating malignancy is detected is found as a result of the interpretation will be described. FIG. 13 is a diagram illustrating an interpretation result. F13A illustrated in FIG. 13 is a finding statement of the interpretation report. As illustrated in F13A, the finding statement describes “A tumor with a long diameter of 3 cm is observed in S4 of the middle lobe of the right lung. Spicules are observed”.

F13B illustrated in FIG. 13 is the structured result of the finding statement of F13A. As illustrated in F13B, as a result of the structuring, the relationship is specified and classified such that “tumor” is in the item of “lesion”, “yes” is in the item of “facticity of lesion”, and “spicule” is in the item of “characteristic”. The spicule is a characteristic indicating malignancy, and the medical information processing apparatus 16 decides that the association of the key image with the finding statement is necessary on the basis of “spicule” of the structured result.

In this manner, the medical information processing apparatus 16 decides that the association of the key image with the finding statement is necessary in a case where the word representing the characteristic indicates malignancy.

### Eighth Example

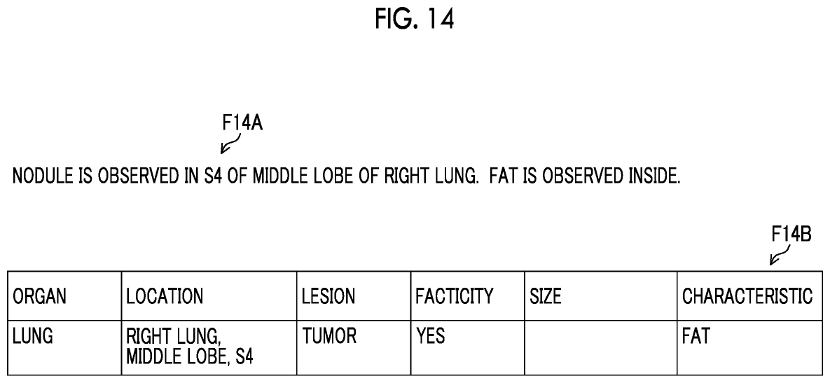

An example in which a lung nodule of which the characteristic indicating benign is detected is found as a result of the interpretation will be described. FIG. 14 is a diagram illustrating an interpretation result. F14A illustrated in FIG. 14 is a finding statement of the interpretation report. As illustrated in F14A, the finding statement describes “A nodule is observed in S4 of the middle lobe of the right lung. Fat is observed inside”.

F14B illustrated in FIG. 14 is the structured result of the finding statement of F14A. As illustrated in F14B, as a result of the structuring, the relationship is specified and classified such that “tumor” is in the item of “lesion”, “yes” is in the item of “facticity of lesion”, and “fat” is in the item of “characteristic”. The fat is a characteristic indicating benign, and the medical information processing apparatus 16 decides that the association of the key image with the finding statement is not necessary on the basis of “fat” of the structured result.

In this manner, the medical information processing apparatus 16 decides that the association of the key image with the finding statement is not necessary in a case where the word representing the characteristic indicates benign.

Structuring

The details of structuring the finding statement by natural language processing will be described with an example.

### Ninth Example

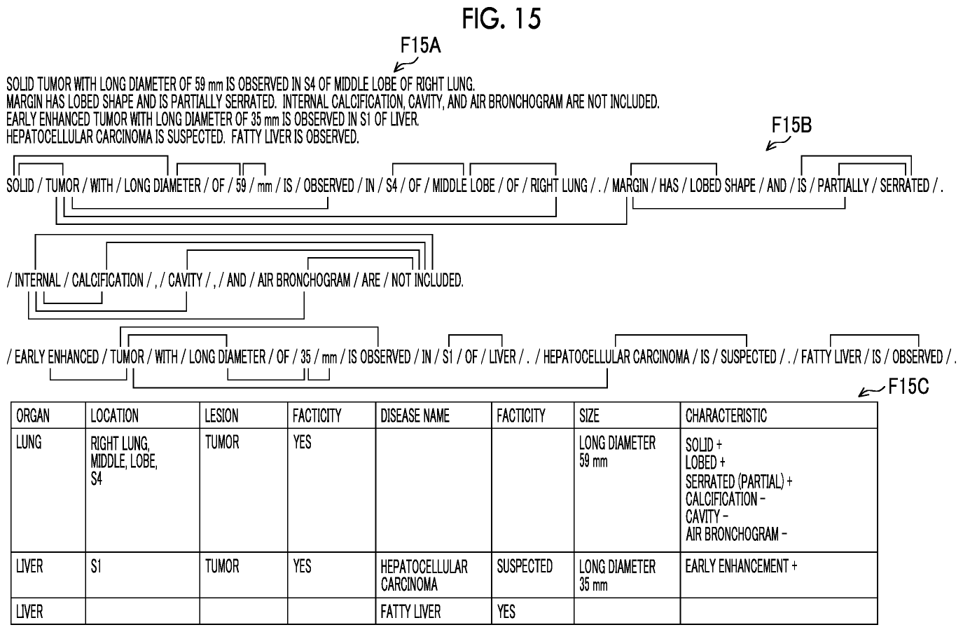

FIG. 15 is a diagram for describing the structuring of the finding statement. F15A illustrated in FIG. 15 is the finding statement of the interpretation report. As illustrated in F15A, the finding statement describes “A solid tumor with a long diameter of 59 mm is observed in S4 of the middle lobe of the right lung. The margin has a lobed shape and is partially serrated. Internal calcification, cavity, and air bronchogram are not included. An early enhanced tumor with a long diameter of 35 mm is observed in S1 of the liver. A hepatocellular carcinoma is suspected. A fatty liver is observed”.

The medical information processing apparatus 16 performs the morphological analysis on the finding statement illustrated in F15A, and converts the finding statement into a word string divided into words. Further, the medical information processing apparatus 16 performs the syntactic analysis on the word string, and specifies the relationship of the words.

F15B illustrated in FIG. 15 is a diagram illustrating the word string converted from the finding statement illustrated in F15A and the relationship extracted from the word string. In F15B, the parts separated by “I” indicate words, and each line connecting the words indicates the specified relationship. Here, the finding statement is converted into word strings “A/solid/tumor/with/a/long diameter/of/59/mm/is/observed/in/S4/of/the/middle lobe/of/the/right lung/. /A/margin/has/a/lobed shape/and/is/partially/serrated/. /Internal/calcification/, /cavity/, /and/air bronchogram/are/not included/. /An/early enhanced/tumor/with/a/long diameter/of/35/mm/is observed/in/S1/of/the/liver/. /A /hepatocellular carcinoma/is/suspected/. /A/fatty liver/is/observed/.”, and further, the relationship of the words is specified.

For example, “right lung” is specified to have a relationship with “middle lobe” and “tumor”. “Tumor” is specified to have a relationship with “right lung”, “solid”, and “observed” in the same sentence, and is further specified to have a relationship with “margin” in the next sentence. In this manner, the relationship is not limited to the words in the same sentence, and is specified also in the words present in the different sentences.

F15C illustrated in FIG. 15 is the structured result of the finding statement using the analysis result illustrated in F15B. Here, the relationship is specified and classified such that “lung” is in the item of “organ”, “right lung”, “middle lobe”, and “S4” are in the item of “location”, “tumor” is in the item of “lesion”, “yes” is in the item of “facticity of lesion”, “59 mm” is in the item of “size”, and “solid+”, “lobed+”, “serrated (partial)+”, “calcification −”, “cavity −”, and “air bronchogram −” are in the item of “characteristic”.

Here, the relationship is specified and classified such that “liver” is in the item of “organ”, “S1” is in the item of “location”, “tumor” is in the item of “lesion”, “yes” is in the item of “facticity of lesion”, “hepatocellular carcinoma” is in the item of “disease name”, “suspected” is in the item of “facticity of disease name”, “long diameter 35 mm” is in the item of “size”, and “early enhancement+” is in the item of “characteristic”. Further, the relationship is specified and classified such that “liver” is in the item of “organ”, “fatty liver” is in the item of “disease name”, and “yes” is in the item of “facticity of disease name”.

### Tenth Example

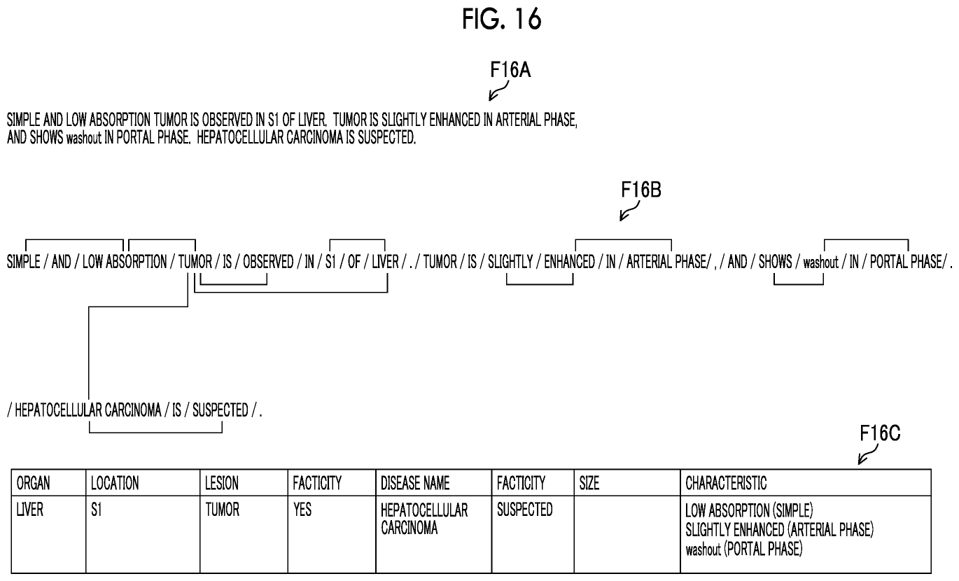

FIG. 16 is a diagram for describing the structuring of the finding statement. F16A illustrated in FIG. 16 is the finding statement of the interpretation report. As illustrated in F16A, the finding statement describes “a simple and low absorption tumor is observed in S1 of the liver. The tumor is slightly enhanced in the arterial phase, and shows washout in the portal phase. A hepatocellular carcinoma is suspected”.

F16B illustrated in FIG. 16 is a diagram illustrating the analysis result by the natural language processing of the sentences illustrated in F16A. Here, the sentences are converted into word strings “A/simple/and/low absorption/tumor/is/observed/in/S1/of/the/liver/. /The/tumor/is/slightly/enhanced/in/the/arterial phase/, /and/shows/washout/in/the/portal phase/. /A/hepatocellular carcinoma/is/suspected/.”, and further, the relationship of the words is specified.

For example, “simple” is specified to have a relationship with “low absorption”, “arterial phase” is specified to have a relationship with “enhanced”, and “portal phase” is specified to have a relationship with “washout”.

F16C illustrated in FIG. 16 is the structured result of the finding statement using the analysis result illustrated in F16B. Here, the relationship is specified and classified such that “liver” is in the item of “organ”, “S1” is in the item of “location”, “tumor” is in the item of “lesion”, “yes” is in the item of “facticity of lesion”, “hepatocellular carcinoma” is in the item of “disease name”, “suspected” is in the item of “facticity of disease name”, and “low absorption (simple)”, “slightly enhanced (arterial phase)”, and “washout (portal phase)” are in the item of “characteristic”.

In this manner, the medical information processing apparatus 16 associates the characteristic with the imaging conditions.

### Eleventh Example

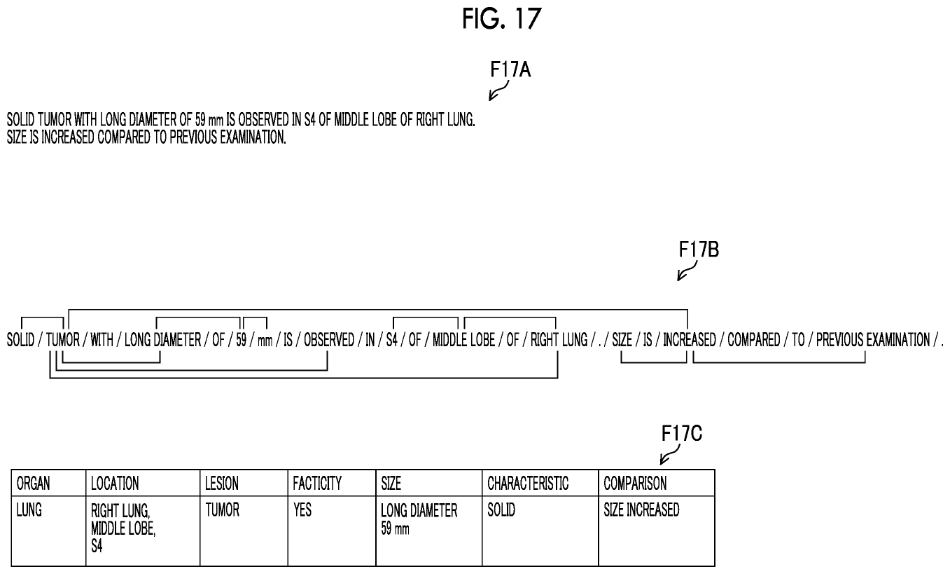

FIG. 17 is a diagram for describing the structuring of the finding statement. F17A illustrated in FIG. 17 is the finding statement of the interpretation report. As illustrated in F17A, the finding statement describes “A solid tumor with a long diameter of 59 mm is observed in S4 of the middle lobe of the right lung. The size is increased compared to the previous examination”.

F17B illustrated in FIG. 17 is a diagram illustrating the analysis result by the natural language processing of the sentences illustrated in F17A. Here, the sentences are converted into word strings “A/solid/tumor/with/a/long diameter/of/59/mm/is/observed/in/S4/of/the/middle lobe/of/the/right lung/. /The/size/is/increased/compared/to/the/previous examination/.”, and further, the relationship of the words is specified.

For example, “increased” is specified to have a relationship with “previous examination” and “size” in the same sentence, and is further specified to have a relationship with “tumor” in the previous sentence. F17C illustrated in FIG. 17 is the structured result of the finding statement using the analysis result illustrated in F17B. Here, the relationship is specified and classified such that “lung” is in the item of “organ”, “right lung”, “middle lobe”, and “S4” are in the item of “location”, “tumor” is in the item of “lesion”, “yes” is in the item of “facticity of lesion”, “long diameter 59 mm” is in the item of “size”, “solid” is in the item of “characteristic”, and “size increased” is in the item of “comparison”.

In this manner, the medical information processing apparatus 16 extracts comparison information.

Others

In the techniques disclosed in JP2015-162082A and JP2018-028562A, there is no option of not associating the key image, but in the present embodiment, it is possible to decide the necessity of the association of the key image based on the image with the series of sentences.

The processing relating to the key image may be decided using the accessory information of the medical image. For example, the accessory information of the medical image may be acquired, the lesion information may be acquired by analyzing the interpretation report, and at least one of the necessity of the association of the key image with the finding statement or the candidates for the key image to be associated with the finding statement may be decided on the basis of the accessory information and the lesion information. The accessory information includes slice intervals, contrast information (non-contrast/contrast, arterial phase/portal phase/equilibrium phase), and the like. The slice interval is the distance between adjacent slice images in a direction orthogonal to the slice direction.

The processing relating to the key image according to the present embodiment can be applied to non-medical images. For example, for social infrastructure facilities such as transportation, electricity, gas, and water, images and series of sentences can be accepted, the relationship of two or more words included in the series of sentences can be specified, and at least one of the necessity of the association of the key image based on the image with the series of sentences or the candidates for the key image to be associated with the series of sentences can be decided on the basis of the relationship of the two or more words.

The technical scope of the present invention is not limited to the scope described in the above embodiments. The configurations and the like in the embodiments can be appropriately combined between the embodiments in a range not departing from the gist of the present invention.

### EXPLANATION OF REFERENCES

10: medical information processing system
12: medical image examination device
14: medical image database
16: medical information processing apparatus
16A: processor
16B: memory
16C: communication interface
18: interpretation report database
20: user terminal
20A: input device
20B: display
22: network
A1: annotation
A2: annotation
ID1˜ID5: diagnostic image
IK1: key image
IK2: key image
RP1: interpretation report
RP2: interpretation report
ST1-ST5: step of medical information processing method

What is claimed is:

1. An information processing apparatus comprising:
at least one processor; and
at least one memory that stores a command for the at least one processor to execute,
wherein the at least one processor is configured to:
accept a series of sentences including a diagnosis result of an image;
specify a relationship of two or more words included in the series of sentences; and
decide at least one of necessity of association of a key image based on the image with the series of sentences or a candidate for the key image to be associated with the series of sentences on the basis of the relationship of the two or more words.

2. The information processing apparatus according to claim 1,
wherein the two or more words include at least two words from among a word representing a region of interest, a word representing facticity, a word representing change information, a word representing a position, a word representing a size, a word representing a characteristic, or a word representing an imaging condition.

3. The information processing apparatus according to claim 1,
wherein the two or more words include a word representing a region of interest, and a word representing facticity of the region of interest, and
the at least one processor is configured to:
determine that the association of the key image is necessary in a case where the facticity affirms existence of the region of interest; and
determine that the association of the key image is not necessary in a case where the facticity denies the existence of the region of interest.

4. The information processing apparatus according to claim 2,
wherein the word representing the change information includes a word representing change information on at least one of a size or an amount.

5. The information processing apparatus according to claim 2,
wherein the at least one processor extracts the candidate for the key image from the image on the basis of the position.

6. The information processing apparatus according to claim 1,
wherein the at least one processor is configured to:
accept two or more types of images of which the imaging conditions are different; and
extract the candidate for the key image from the two or more types of images.

7. The information processing apparatus according to claim 1,
wherein the image is a medical image,
the two or more words include a word representing a disease name, and
the at least one processor extracts the candidate for the key image on the basis of the disease name.

8. The information processing apparatus according to claim 1,
wherein the two or more words include a word representing a region of interest, and a word representing a malignancy grade of the region of interest, and
the at least one processor is configured to:
determine that the association of the key image is necessary in a case where the malignancy grade affirms malignancy of the region of interest; and
determine that the association of the key image is not necessary in a case where the malignancy grade denies the malignancy of the region of interest.

9. The information processing apparatus according to claim 1,
wherein the at least one processor is configured to:
display the candidate for the key image on a display;
accept an operation by a user; and
associate the candidate of the key image with the series of sentences, as the key image according to the operation.

10. The information processing apparatus according to claim 1,
wherein the image is a three-dimensional image, and
the at least one processor is configured to:
display, as the candidate for the key image, a slice image at any slice position of the three-dimensional image on a display;
accept a change of the slice position of the candidate for the key image by a user; and
associate the slice image at the changed slice position with the series of sentences, as the key image.

11. An information processing method comprising:
an acceptance step of accepting a series of sentences including a diagnosis result of an image;
a specifying step of specifying a relationship of two or more words included in the series of sentences; and
a decision step of deciding at least one of necessity of association of a key image based on the image with the series of sentences or a candidate for the key image to be associated with the series of sentences on the basis of the relationship of the two or more words.

12. A non-transitory, computer-readable tangible recording medium which records thereon a program for causing, when read by a computer, the computer to execute the information processing method according to claim 11.
